(19) United States
(12) Patent Application Publication
(10) Pub. No.: US 2014/0275511 A1
(43) Pub. Date: Sep. 18, 2014

(54) PURIFICATION OF NIKKOMYCIN Z

(71) Applicant: David J. Larwood, Woodside, CA (US)

(72) Inventor: David J. Larwood, Woodside, CA (US)

(21) Appl. No.: 13/843,829

(22) Filed: Mar. 15, 2013

(51) Int. Cl. C07H 19/06

(52) U.S. Cl. CPC C07H 19/06
USPC 536/28.53

(57) ABSTRACT

This invention relates to an improved method of purifying Nikkomycin Z, an antifungal compound shown to be particularly useful in treating Valley Fever, coccidioidomycosis. The improvement of this invention lies in the choice of resins and elution conditions, chosen to minimize handling of the material.

### FIELD OF THE INVENTION

This invention relates to an improved method of purifying Nikkomycin Z, an antifungal compound shown to be particularly useful in treating Valley Fever, coccidioidomycosis. The process involves purifying fermentation broth containing Nikkomycin Z on a cation exchange resin, eluting with a suitable base, purifying on an anion exchange resin, eluting with a suitable acid, and separating undesired organic compounds on a HIC column. The product peaks are acidified as a suitable salt and isolated by drying, such as by lyophilization. The improvement of this invention lies in the choice of resins and elution conditions, chosen to minimize handling of the material.

### BACKGROUND OF THE INVENTION

Valley Fever (Coccidioidomycosis) has been known since 1882. The history of the disease and how it has been identified by medical scientists has been detailed. See, for example, Drutz D J, Catanzaro A. Coccidioidomycosis. Part I. Am Rev Respir Dis. 1978; 117:559-85, and Drutz D J, Catanzaro A. Coccidioidomycosis. Part II. Am Rev Respir Dis. 1978; 117:727-71.

A recent review gives a good perspective. “An insight into the antifungal pipeline: selected new molecules and beyond.” by L Ostrosky-Zeichner; A Casadevall; J N Galgiani; F C Odds; J H Rex; Drug discovery, 2010 September; 9(9): 719-27.

See also the introductory remarks in a 2007 compendium of the decennial meeting of the Coccidioidomycosis Study Group, “Coccidioidomycosis: Changing Perceptions and Creating Opportunities for Its Control,” John N. Galgiani, Ann. N.Y. Acad. Sci. 1111: 1-18 (2007), New York Academy of Sciences.

Nikkomycin Z (“Nik Z”) was identified as an active compound by Bayer Pharmaceuticals in the 1970s. See U.S. Pat. Nos. 4,046,881 and 4,158,608 (Daehn et al.). Bayer and others filed several patents regarding Nikkomycin Z, analysis, and isolation of the product. In 1981, Bayer was issued a patent on purifying Nikkomycin compounds. U.S. Pat. No. 4,287,186 describes culturing a production strain of Streptomyces tendae TU 901, filtering, and loading onto a column of Dowex Wx4 (50-100 mesh, Na+form). The column was washed with water until eluent is colorless, then eluted with dilute ammonia water. The selected fractions were evaporated to release ammonia, acidified, then eluted on an Amberlite 252 column. This was again washed with water, then eluted with ammonia water, concentrated and acidified. After an anion exchange column (SP-Sephadex-25) and a neutral column (Bio-Gel P 2), the biologically active fractions were combined and lyophilized. The patent describes an hplc assay. See also U.S. Pat. No. 4,402,947 (Moeschler et al.), assigned to Bayer.

A later patent, U.S. Pat. No. 4,552,954 (Moeschler et al.), assigned to Bayer, cites these two patents and other references, noting that the process of '186 is multistep and commercially unsatisfactory.

Nikkomycin Z was tested against Valley Fever and found to be quite effective at treating infected animals. Hector, R. F., Zimmer, B. L. & Pappagianis, D. Evaluation of nikkomycins X and Z in murine models of coccidioidomycosis, histoplasmosis, N and blastomycosis. Antimicrob. Agents Chemother. 34, 587-593 (1990).

Shaman Pharmaceutical continued work on Nikkomycin Z, for Valley Fever and other indications, filing an IND and conducting initial human trials with a single ascending dose trial. Nix, D. E., Swezey, R. R., Hector, R. & Galgiani, J. N. Pharmacokinetics of nikkomycin Z after single rising oral doses. Antimicrob. Agents Chemother. 53, 2517-2521 (2009).

The Shaman technology package was transferred to the University of Arizona in 2005 and has since been developed by the University and Valley Fever Solutions, Inc. L Ostrosky-Zeichner, et al., Drug discovery, 2010 September; 9(9): 719-27.

The University of Arizona contracted with the University of Minnesota to grow a modified S. tendae organism, modified to minimize production of Nikkomycin X. Valley Fever Solutions then contracted with the University of Minnesota to repeat the Shaman recovery process. That group extended the Shaman three column process to a five column process. See Stenland et al., Org. Process Res. Dev., 2013, 17, 265-272. The HPLC assay reported in that article is essentially the one Valley Fever Solutions shared with Minnesota, as developed at SRI, Menlo Park, Calif. That SRI assay has been used by several of our contractors. SRI used a Phenomenex Gemini C18 3 micron column, 150×4.6 mm monitoring UV at 280 nm, bandwidth 16 nm.

### DESCRIPTION OF THE INVENTION

### Summary

We have improved upon known purification conditions to use a simpler flow, with fewer processing steps than in the prior art.

Briefly, we elute the cation exchange column with a moderately concentrated base, so as to release the adsorbed material without undue salt. This is processed quickly to a pH suitable for loading on the anion column.

The anion-adsorbed material is eluted with a moderately acidic eluent. It is desirable to get fractions that are reasonably concentrated but not too salty so they can be used to load the HIC column directly.

The detailed description elaborates on the conditions.

### DESCRIPTION OF THE DRAWINGS

-no drawings-

### DETAILED DESCRIPTION OF THE INVENTION

We have reviewed the Shaman three column process and made improvements thereon. In basic outline it is similar to the early Bayer work. Choosing materials and conditions more selectively permits the process to be run in a more practical and efficient manner.

For source material, the University of Arizona construct Streptomyces tendae Delta-NikQ 25-2 as modified at the University of Minnesota (UMNO33G2) is useful. See Stenland et al., Org. Process Res. Dev., 2013, 17, 265-272. Minnesota ran a sizable fermentation (350 L) in 2010, from which the broth was frozen and used for two years. They made another similar run in September 2012 with a bit higher yield in the fermentation. This material also was frozen. Freezing the broth brings the NikZ titer down to on the order of 0.7-0.9 mg NikZ/mL. Purifying broth from these two fermentation has yielded at least similar results.

The basic process from there has some similarities to the Bayer and Minnesota descriptions, with the following details.

First, the fermentation broth is preferably adjusted in pH to 4.0±0.5. Bayer recommends using acetic acid. We have found this useful. Broth which is frozen and later thawed will tend to lose some NikZ potency. Broth which is at a pH of 6 will tend to degrade, even in the refrigerator, if held more than some few hours, and certainly over multiple days. At pH 4 it seems to be reasonably stable for at least a week at 2-8° C. It is preferable to use fresh broth.

Filtration with TFF (Tangential Flow Filtration) is advantageous. Broth can be filtered through a 3 KD exclusion filter but this is slow. Filtering through a 5 KD membrane gave a reasonable balance of exclusion and speed. Novasep 5 kD Hystream, 0.1 m². We have had good success using a 10 KD membrane, and that is our current preferred process. In a preferred embodiment, the frozen broth is filtered through a 0.2 micron and then a 0.1 micron filter shortly prior to TFF processing. This gives on the order of 90% recovery and a significant decolorization of the broth.

This material is loaded on a suitable cation exchange (CEX) column, then washed well with water. 5 to 10 column volumes was common. Monitoring UV at 280 nM, there is a large rise with product load and washout, which decreases with water wash, approaching baseline, often under 1 AU (absorbance units) or even 0.5 AU.

We have used DowexW50×4 (100-200 mesh), DowexW×4 (50-100 mesh) and DowexW50×2 (50-100 mesh) with good results. Bayer reports a wide range of similar resins. One skilled in the art will recognize that a broad range of such resins are candidates for this process.

We eluted with a variety of mild bases. The Shaman process uses 0.2 N ammonium hydroxide. Bayer teaches using 0.1 N ammonium hydroxide, and preferably less Although we got some resolution at the lower 0.1N level, we found that 0.15 N ammonium hydroxide gave reasonably fast elution without an unduly high amount of salt in the eluted fractions. We also used 0.15N NaOH to good effect. Using 0.1N NaOH eluted the product, but with a long, slow tail containing about 10% of product. This tail was considerably shortened when using 0.15N NaOH.

At this point, Bayer and Shaman teach acidifying the cation eluate to pH 4. This is a useful step if the material is to be held, as it is not stable for long in the moderate to high pH cation fractions. The AEX column preferably is loaded with material at pH about 8.0. As the product is unstable below pH 6, and most stable in the range pH 3-4, adjusting pH above about 5 should be done only shortly before the material is needed at any elevated a pH range.

For our preferred process, we take the CEX fractions, some of which are at pH 11 and thus quite sensitive., and process them quickly for AEX loading. We pooled fractions and quickly loaded it onto an anion exchange column, without this acidification step. Before loading on AEX, we diluted the material to keep conductivity under 15 mS/cm, and adjusted the pH to 8.0±0.5, checking again the conductivity. Since the anion resin will bind the product, this can be moderately dilute. The native CEX eluate ranged from 0.1 to 7 mg Nikkomycin Z/mL. Pooled, the peak was typically on the order of 3-4 mg/mL. Diluted for anion load, and adjusted in pH, the load was often on the order of 2 mg/mL. The process seems to be useful with a load of 1 mg/mL, and we expect it to be useful at lower concentrations. Fractions are on the order of up to 8 mg NikZ/mL. A 30 mL CEX column will process 1 L of broth well.

For anion exchange (AEX) resin, Bayer teaches a wide range of candidate materials. We were successful with Dowex1×4 (varying mesh sizes) and Dowex 1×2 (50-100 mesh). Recently, we have used DEAE-Sephadex-A25 to good effect.

After loading material on the AEX resin, we wash with water. As with the CEX resin elution, an early UV 280 nm peak diminishes with the water wash over some 5 to 10 column volumes of water. Elution can be with a gradient. We have had good success with a step to 0.1 N HCl. A higher step to 0.2 N HCl gives a sharper, more concentrated peak. It however tends to have a higher salt concentration, which can interfere with the subsequent HIC column performance. We prefer 0.1 to 0.15 N HCl for this elution.

A 20 mL AEX column is more than enough capacity for a corresponding 30 mL CEX column. We often use column volume ratios of 2:1:2 for CEX:AEX:HIC.

For a hydrophobic interaction chromatography (HIC) column, we used the Shaman recommendation of Amberchrom cg-161m. The resin has a capacity of on the order of 10 mg NikZ/mL resin, and reportedly up to 15 mg/mL resin. Load volumes greater than 1.1 CV can be problematic. Concentrating the load, either by direct AEX fraction concentration, or by concentration of AEX fractions, allows a compact load for the HIC column. We have had good success with concentrating AEX fractions to 20 mg NikZ/mL. We have had good HIC performance loading direct AEX elutions at a concentration as low as 2 mg NikZ/mL. HIC will resolve the final products with even lower loading concentration (0.5 mf NikZ/mL has been successful). However this does not use the capacity of the resin and the product elutes at correspondingly low concentration. A load of 0.5 CV at a concentration of 20 mg NikZ/mL is desirable. This is 10 mg NikZ in 1 CV, which is a useful load on the resin. The load amount can be increased by up to about 50% when the column is running well. Thus 20 mg NikZ/mL in 0.6 to 0.7 CV is an excellent load, and correspondingly less volume if the material is more concentrated.

The HIC column is equilibrated with 100 mM Sodium phosphate, pH 6.0 for 1 to 3 CV. It can be washed with water before loading. We prefer to step down the equilibration buffer to 10 mM for 1 to 3 CV, then load the product without a water wash. The load material is adjusted to pH 6.0±0.25 shortly before loading.

Elution is with water. After loading and early washing, we typically see a rise in UV about 1 CV after loading is initiated. This typically goes down again before rising at about 3 CV after starting the load. There is often a dip in pH after loading ((the load materials is at pH 6), then a rise to on the order of 7, then decreasing again. This almost always starts decreasing well before the product elutes. Product elutes typically over about 2 CV, from about 3.5 CV after loading to about 5.5 or 6 CV after load starts. As there are some impurities in both the leading edge of the product peak and the tail, our preferred collection is 0.25 to 0.3 CV fractions until the product peak clearly starts, 1 CV central fraction, 0.5 post central fraction, then two 05 CV tail fractions. With experience in a particular system this number of fractions can be reduced.

The leading edge fractions and tail fractions deemed not sufficiently pure can be retained. These can be purified again with this same process over the AEX and HIC steps.

HIC fractions are typically better than 98% pure by hplc. Selected fractions are pooled, acidified with HCl to pH 3.5±0.3 (preferably pH 3.4±0.1). This material, if more than 1 mg NikZ/mL, can be spray dried directly. If the load material is sufficiently concentrated, these concentrations can easily be 5 or even 15 mg nikZ/mL, which is quite suitable for spray drying.

An alternative method of concentration is by rotary evaporation. It is highly preferable to use a high vacuum, better than 29 inches of mercury. Less than 50 milliTorr is desirable, and particularly 10-20 milliTorr. Bath temperature should be 35° C. for the first 50% volume reduction, then can be raised to 45° C. With experience, these temperatures can be increased a bit. When suitably concentrated, the material can be lyophilized. Drying in a vacuum oven at up to 60° C. for 24 hours should get the moisture to less than 6%. 3 to 4% moisture is preferable. The material can be stored in a well closed vessel for long periods. A well stoppered brown bottle, at 2-8° C. can keep the material for years without undue degradation.

### Conditions and Recovery Summary:

Dowex50Wx2 30 mL load 732 mg in 1 L broth (24 mg NikZ/mL resin)
Elutes with 0.15N NH₄OH
Recovery 515.4 mg (70%) in the main peak, plus 78 mg (11%) in a later fraction
   DEAE-Sephadex-A25 20 mL Load 515 mg in 30 mL pH adjusted to 8.0 (26 mg NikZ/mg resin)
   Elute with 0.1N HCl
Recovery 460 mg (89%) in a main peak, and another 52 mg (10%) in a later fraction
   Amberchrom cg-161-m 160 mL Load 400 mg in 81 mL, pH 6.2 (5 mg NikZ/mL resin)
   Elute with water
Recovery 238 mg (59%) at >99% purity, plus 130 mg (32%) at >95% purity
   Overall: 32.4% 99% pure (from starting broth).

We are working to increase this recovery by more thoughtful peak cuts in the CEX and AEX columns.

The preferred embodiments described herein are illustrative only, and although the examples given include many specifications, they are intended as illustrative of only a few possible embodiments of the invention. Other embodiments and modifications will occur to those skilled in the art. The examples given should only be interpreted as illustrations of some of the preferred embodiments of the invention, and the full scope of the invention should be determined by the appended claims and their legal equivalents.

What is claimed is:

1. A process for concentrating Nikkomycins Z from a dilute impure aqueous solution thereof containing Nikkomycin Z comprising
bringing the solution to a pH between about 4 and 7,
contacting the solution with a suitable cation exchange resin,
eluting the adsorbed material with a mild base solution,
transferring eluate of interest and contacting with a suitable anion exchange resin,
eluting the adsorbed material with a mild acid solution,
transferring eluate of interest to a HIC column,
eluting the material with water,
thereby to obtain a concentrated and purified solution of Nikkomycin Z.
